(19) United States
(12) Patent Application Publication
(10) Pub. No.: US 2025/0249280 A1
(43) Pub. Date: Aug. 7, 2025

(54) SYSTEM AND METHOD FOR OPTICAL STIMULATION

(71) Applicant: Science Corporation, Alameda, CA (US)

(72) Inventors: Elizabeth Carroll, Alameda, CA (US); Max Hodak, Alameda, CA (US); Alan Mardinly, Alameda, CA (US); Yifan Kong, Alameda, CA (US); Antonia Elsen, Alameda, CA (US)

(73) Assignee: Science Corporation, Alameda, CA (US)

(21) Appl. No.: 19/044,388

(22) Filed: Feb. 3, 2025

Related U.S. Application Data
(60) Provisional application No. 63/548,591, filed on Feb. 1, 2024.

(51) Int. Cl. A61N 5/06

(52) U.S. Cl. CPC A61N 5/0622; A61N 2005/063; A61N 2005/0658; A61N 2005/0666

(57) ABSTRACT

The system for optical stimulation can include: a light source, a display module, a steering module, a sensor module, and a processing system. In variants, the system can function to project a time series of patterned light frames onto the retina of an eye (e.g., onto optogenetically modified cells in the retina, onto a retinal implant, etc.).

### CROSS-REFERENCE TO RELATED APPLICATIONS

This application claims the benefit of U.S. Provisional Application No. 63/548,591 filed 01 Feb. 2024, which is incorporated in its entirety by this reference

### TECHNICAL FIELD

This invention relates generally to the optics field, and more specifically to a new and useful system and method for optical stimulation in the optics field.

### BRIEF DESCRIPTION OF THE FIGURES

FIG. 1 is a schematic representation of a variant of the system.

FIG. 2 is a schematic representation of an example of the system.

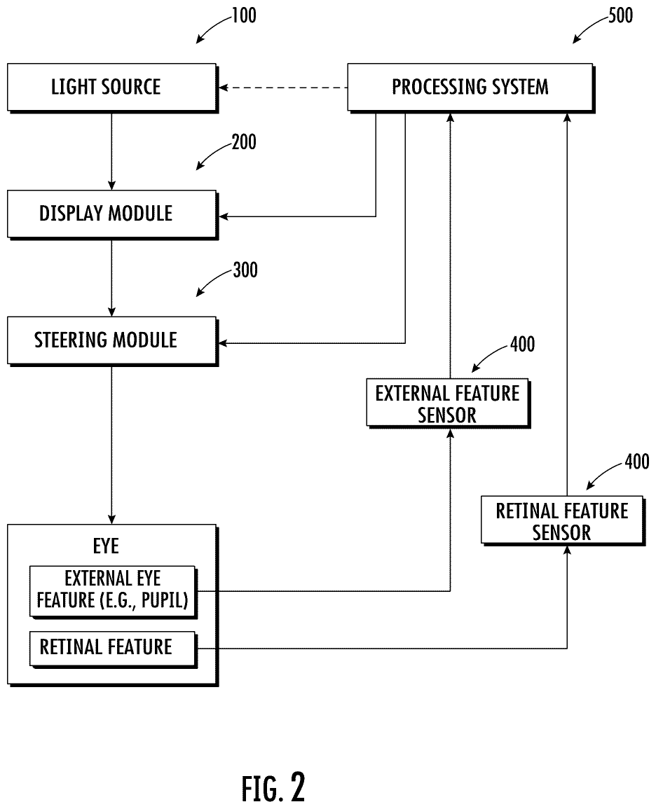

FIGS. 3A and 3B depicts specific examples of the system.

FIG. 4A depicts a first illustrative example of the system.

FIG. 4B depicts a second illustrative example of the system.

FIG. 5 is a schematic representation of a specific example of the system.

FIG. 6A depicts an example of a display module, including a spatial light modulator (SLM).

FIG. 6B depicts another example of a display module, including a spatial light modulator (SLM).

FIG. 7 depicts an example of steering the output light.

### DETAILED DESCRIPTION

The following description of the embodiments of the invention is not intended to limit the invention to these embodiments, but rather to enable any person skilled in the art to make and use this invention.

### 1. Overview

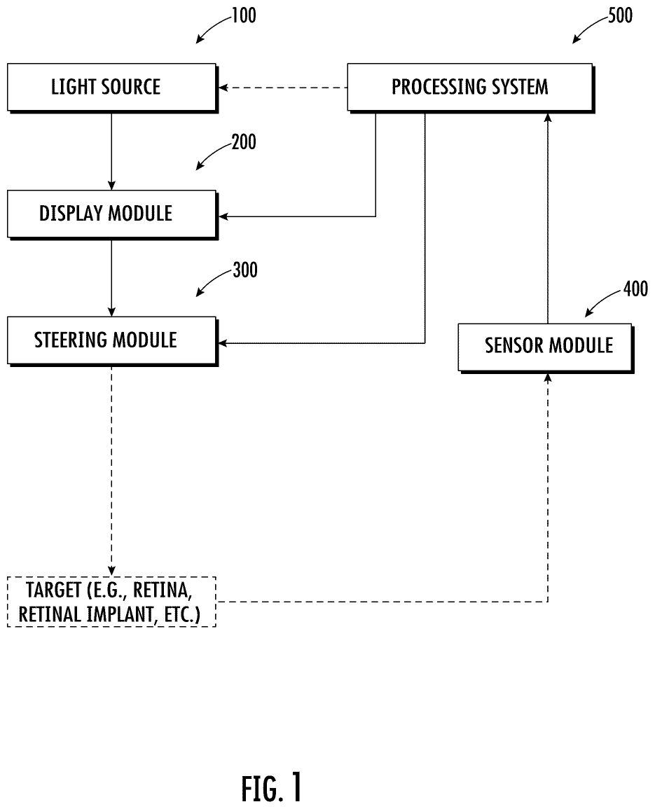

As shown in FIG. 1, the system can include: a light source 100, a display module 200, a steering module 300, a sensor module 400, and a processing system 500. However, the method can additionally or alternatively include any other suitable components.

In variants, the system can function to project a time series of patterned light frames onto the retina of an eye. For example, the system can function to restore vision for users who have lost function of some or all of their photoreceptors (e.g., degenerated and/or otherwise impaired photoreceptors), ganglia, and/or other sensory cells. In a first example, the system functions to optically stimulate (e.g., activate) optogenetically modified cells in the retina. In a second example, the system functions to optically stimulate a retinal implant, wherein the retinal implant stimulates (e.g., activates) retinal cells.

### 2. Examples

In an example, the system can include a projector, including a light source, a spatial light modulator, and a steering module (e.g., an actuated mirror). The projector can project a time series of patterned light frames (e.g., encoding content) towards a retina of a user. In a first specific example, the patterned light frames can be projected onto optogenetically modified cells in the retina, wherein resulting signals from the optogenetically modified cells can be interpreted by the brain (e.g., such that the user can visualize the content). In a second specific example, the patterned light frames can be projected onto a retinal implant, wherein resulting electrical signals generated by the retinal implant can be delivered to native retinal cells and subsequently interpreted by the brain (e.g., such that the user can visualize the content).

In an example, the system can include one or more sensors (e.g., cameras, photodiodes, etc.), which can be used to track one or more external eye features (e.g., pupil, corneal reflection, etc.,), one or more retinal features (e.g., an individual retinal cell, vasculature, retinal implant features, features of the patterned light frame reflecting off the retina), and/or any other target features. These tracked target features can be used to control the steering module to steer output light from the projector, to control one or more spatial light modulators to adjust the patterned light frames, and/or can be otherwise used. In a specific example, a tracked external eye feature (e.g., pupil) can be used to determine an approximate target location, wherein the steering module can be controlled to project the patterned light onto the approximate target location. In this specific example, a tracked retinal feature can then be used to determine a precise target location, wherein the precise target location can be used to adjust the steering module and/or display module (e.g., such that the patterned light frames are projected onto the precise target location).

### 3. Technical Advantages

Variants of the technology can confer one or more advantages over conventional technologies.

First, variants of the technology can restore and/or augment sight in users (e.g., users with blindness). In some diseases like retinitis pigmentosa and macular degeneration, the photoreceptors are damaged while the cells of the optic nerve, the retinal ganglion cells (RGCs), remain functional. Variants of the technology can include a projector that emits patterned light to stimulate cells in the retina (e.g., via stimulation of genetically modified cells in the retina transfected with a gene for a light-sensitive protein and/or via stimulation of a retinal implant). In a specific example, variants of the technology can restore and/or augment visual input to the brain.

Second, variants of the technology can dynamically adjust the projected light to account for eye movement. In variants, this can enable a user to perceive visual content at a high acuity. In a first example, the system can dynamically steer (e.g., in real time) the patterned light projection in response to tracked movement of the eye of the user. In a second example, the system can track retinal features at or near cellular resolution, wherein light pattern parameters (e.g., the direction of the projected light, the light pattern itself, etc.) can be adjusted in response to the tracked retinal features. In an illustrative example, the system can determine and track individual cells in the retina, wherein these tracked cells can be used to control the light pattern parameters. In a specific example, controlling the steering can be used for positioning the patterned light frame at a target location (e.g., at a low spatial resolution), while adjusting the light patterned parameters can be used to perform further, fine-tuned pose adjustments (e.g., orientation, scale, any affine transformation, etc.) to correct for system aberrations and/or aberrations of the eye, enabling higher resolution mapping between the components of the patterned light frame and retinal cells.

Third, variants of the technology can be used for diagnostics. In a first example, retinal images (e.g., fundus images) acquired using the system can be used to evaluate the cells of the retina. In a first specific example, the retinal images can be used to evaluate optogenetically modified cells in the retina (e.g., determining which cells were successfully transfected with the target gene, monitoring the production of the opsin over time, etc.). In a second specific example, the system can be used to perform fundus autofluorescence imaging, where the retinal images can be used to monitor fluorophores in the retina for evaluation of the health of the retina. In a second example, retinal images acquired using the system can be used to evaluate the retinal implant. In an illustrative example, the retinal images can be used to visualize the retinal implant to monitor the location of the retinal implant on the retina.

However, further advantages can be provided by the system and method disclosed herein.

### 4. System

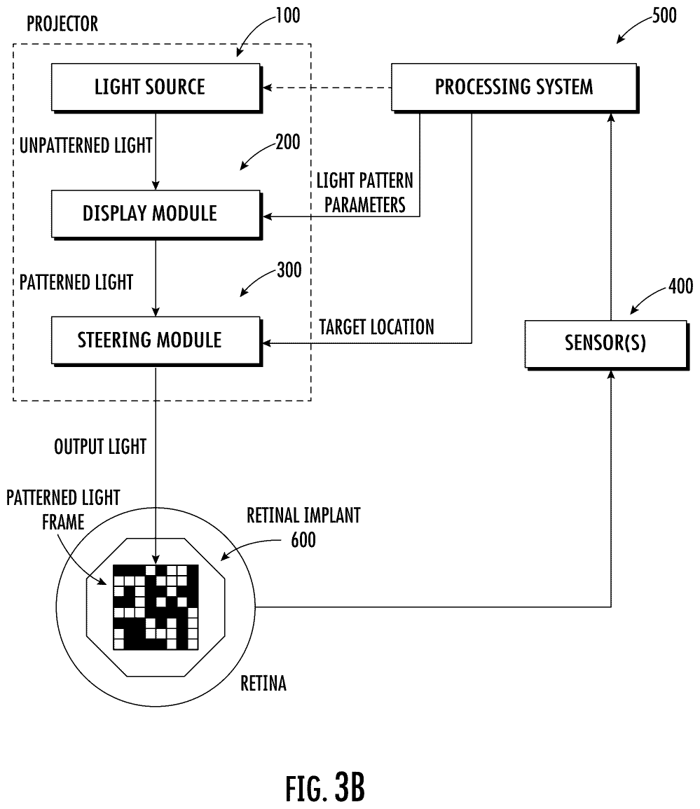

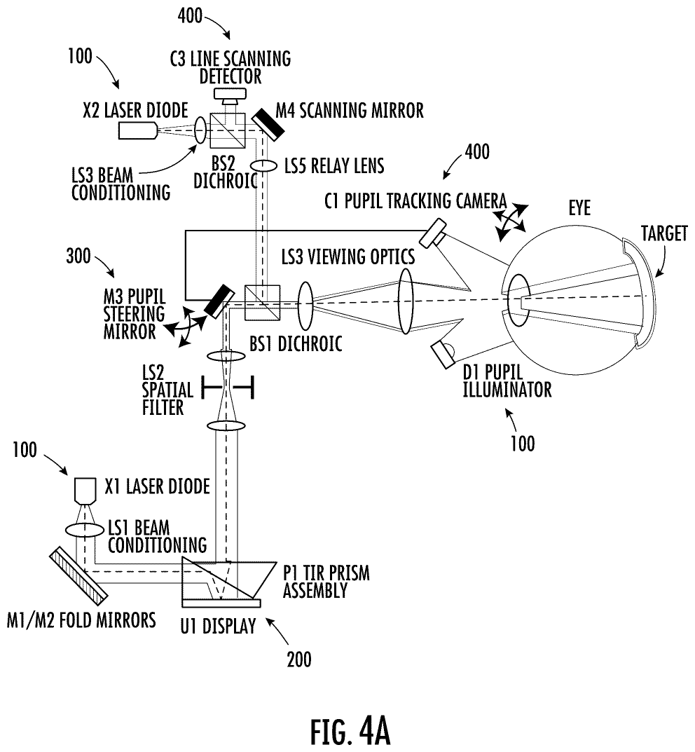

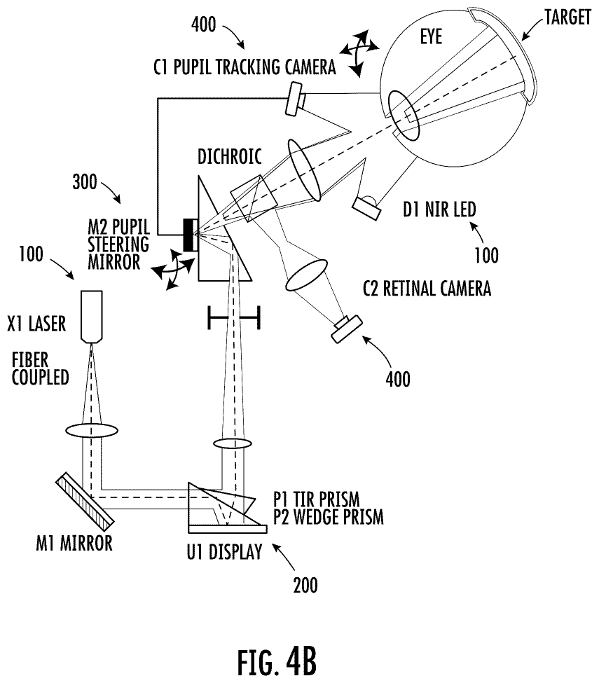

As shown in FIG. 1, the system can include: a light source 100, a display module 200, a steering module 300, a sensor module 400, and a processing system 500. The system can optionally include a retinal implant 600 and/or any other suitable components. Specific examples of the system are shown in FIG. 4A, FIG. 4B, and FIG. 5.

The system can be used with one or more targets. The target can be or include one or more eyes or component(s) thereof (e.g., pupil, fundus, retina, retinal cells, etc.). In a specific example, the target can be a portion of the retina. Additionally or alternatively, the target can include a retinal implant 600 or a component thereof (e.g., pixels of the retinal implant 600). However, the target can additionally or alternatively include another body part of a user (e.g., brain) and/or any other target.

In a first variant, the target can include one or more pixels (e.g., photovoltaic cells, photodiodes, etc.) of a retinal implant 600. In a second variant, the target can include one or more unmodified cells. For example, the target can include one or more unmodified retinal cells (e.g., unmodified retinal ganglion cells). In a third variant, the target can include one or more modified cells. For example, the target can include one or more modified retinal cells (e.g., modified retinal ganglion cells). In an example, the percentage of cells in the target that are modified can be between 5%-100% or any range or value therebetween (e.g., at least 15%, at least 20%, at least 25%, at least 30%, at least 40%, at least 50%, etc.), but can alternatively be less than 5%. In an example, cells in the target can be genetically modified by transfecting the cells with a light-sensitive protein (e.g., using a virus with a plasmid and capsid) that acts as an optogenetic actuator (e.g., optogenetic effector). Optogenetic actuators can produce a biochemical signal (e.g., an action potential) in response to receiving light at a specific wavelength. However, optogenetic actuators can be otherwise defined. The capsid can be an adeno-associated virus capsid (e.g., AAV2.7M8) and/or any other suitable capsid. The plasmid can be an opsin, a fluorescent biosensor protein, and/or any other suitable plasmid. Opsin examples include: CheRiff, ChroMD, ChroME, ChroME2S, ChRmine (e.g., ChRmine-mScarlet), ChrimsonR (e.g., including red-shifted variants), ReachR, and/or any other opsin. In an illustrative example, the cells can be transfected using: AAV2.7m8 hSyn1-ChRmine-Kv2.1-WPRE. Fluorescent biosensor protein examples include: GCaMP8s, GCaMP8m, jRGeco1a, YCaMP, iGECI, and/or any other suitable protein. The opsin can be activated by blue light, green light, red light, and/or any other wavelength.

The system can optionally include one or more optical components. Examples of optical components include: light sources, mirrors, lenses, light modulators, diffractive components, diffusers, filters, fibers, waveguides, prisms, dichroics (e.g., dichroic mirrors), irises, apertures, and/or any other optical components. Optical components can optionally include adaptive (e.g., actuated) optical components.

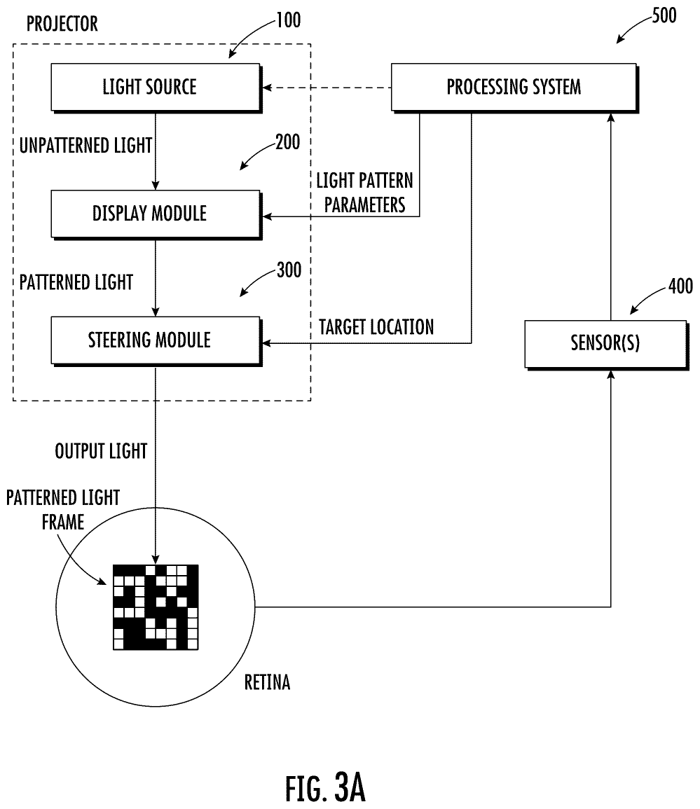

The system can optionally include a projector, wherein the projector includes a set of one or more optical components, including the light source 100, the display module 200, and the steering module 300. Examples are shown in FIG. 3A and FIG. 3B. The projector can project a time series of patterned light frames onto the target (e.g., the retina, the retinal implant 600, etc.). An output light path can define the optical path of output light from the projector (e.g., from a final optical component of the projector) to a target location. In an example, the output light path can include an origin location (e.g., location of the final optical component of the projector) and the target location. The target location can be the location of the target (e.g., the center of the target), a region encompassing the target (e.g., a region of the retina that includes the target), and/or a location and/or region within the target (e.g., a target location on the retina, a target location on the retinal implant 600, etc.). In a first specific example, the target location can be the location of the center of the retina. In a second specific example, the target location can be the location of the center of the retinal implant 600. In a third specific example, the target location can be a region of the retina that includes genetically modified cells. In a fourth specific example, the target location can be the center of the pupil.

### 4.1. The Light Source 100

The light source 100 functions to emit light. The system can include one or more light sources (e.g., at least two light sources, at least three light sources, etc.). The light source 100 can transmit light to one or more optical components (e.g., the display module 200, a mirror, a filter, etc.), transmit light to the target, and/or otherwise interface with the target and/or one or more system components. In a first example, the light source 100 can transmit light to the display module 200 (e.g., directly or via one or more optical components). In a second example, the light source 100 can transmit light to the retina (e.g., directly or via one or more optical components). In a third example, the light source 100 can transmit light to the pupil (e.g., directly or via one or more optical components). In a fourth example, a combination of one or more of the previous examples can be implemented (e.g., using multiple light sources).

The light emitted by the light source 100 is preferably unpatterned (e.g., uniform) light, but can alternatively be patterned light (e.g., wherein the display module 200 includes the light source 100). Examples of light sources include LEDs, μLEDs, lasers (e.g., fiber coupled lasers, laser diodes, etc.), lamps (e.g., incandescent lamps), and/or any other light source. The light can include visible light, UV light, IR light (e.g., NIR light), and/or any other light wavelength. The wavelength of the light can be between 200 nm-5000 nm or any range or value therebetween (e.g., 500 nm-600 nm, 800-2500 nm, at least 500 nm, at least 800 nm, etc.), but can alternatively be less than 200 nm or greater than 5000 nm. In a specific example, when multiple light sources are used, the wavelength of the light for each light source 100 can differ by at least a threshold value (e.g., the wavelengths can differ by at least 100 nm, at least 500 nm, at least 1000 nm, etc.).

However, the light source 100 can be otherwise configured.

### 4.2. The Display Module 200

The display module 200 functions to output patterned light. The display module 200 can receive light from the light source 100, receive light from one or more optical components (e.g., the light source 100 transmits light to a mirror, wherein the light is redirected to the display module 200), transmit light to the steering module 300, transmit light to one or more other optical components, generate light (e.g., the display module 200 includes a light source 100), and/or otherwise interface with one or more system components.

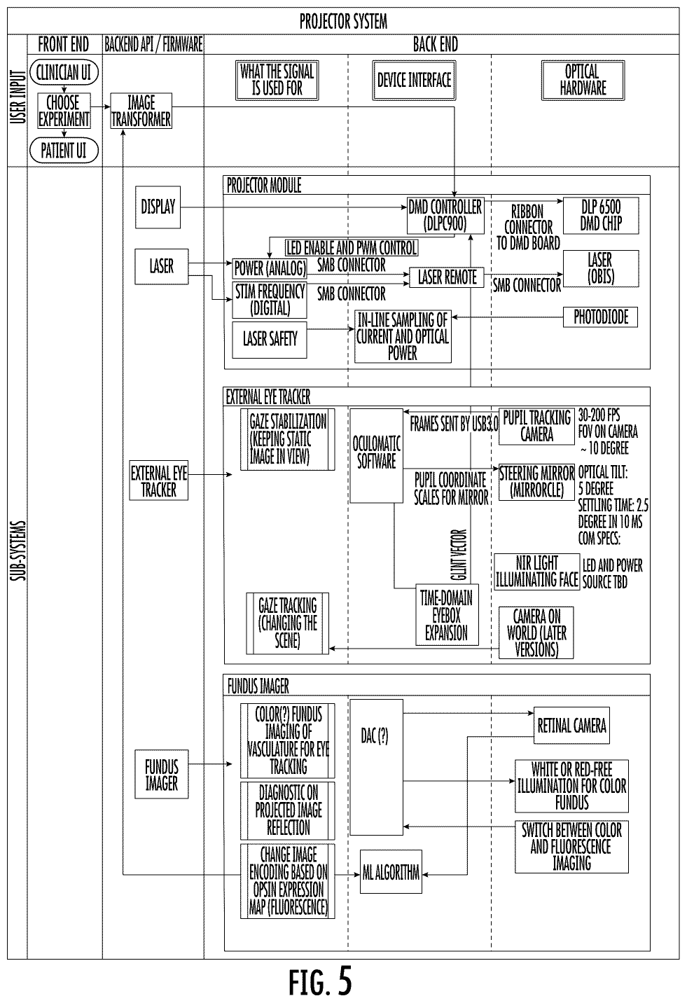

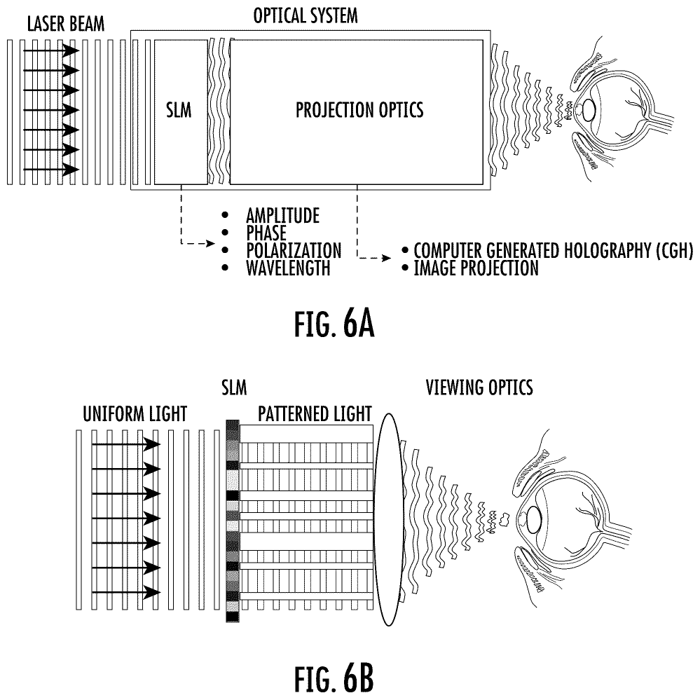

In a first variant, the display module 200 can be or include the light source 100, wherein the display module 200 can emit patterned light. In a second variant, the display module 200 modulates light (e.g., uniform light) received from the light source 100 to produce the patterned light. In an example, the display module 200 can include a spatial light modulator (SLM). Examples are shown in FIG. 6A and FIG. 6B. In specific examples, the display module 200 can include a MEMS-based SLM (e.g., a digital micro-mirror device (DMD)), a LC-based SLM, and/or any other type of SLM. The SLM can be an amplitude modulator and/or a phase modulator. The SLM preferably includes a binary SLM, but can additionally or alternatively include a non-binary SLM. In a specific example, the SLM includes a 1-bit binary amplitude modulator. The SLM can operate between 100 FPS-100,000 FPS or any range or value therebetween (e.g., 10,000 FPS-50,000 FPS; 32,000 FPS; at least 10,000 FPS; at least 20,000 FPS; at least 30,000 FPS; etc.).

The display module 200 can optionally include one or more optical components. For example, the display module 200 can optionally include one or more prisms. In a first example, a prism (e.g., wedge prism) can function to redirect unpatterned light (e.g., from the light source 100, from an optical component, etc.) onto the display module 200. In a second example, a prism (e.g., total internal reflection (TIR) prism) can function to redirect patterned light from the display module 200 to another system component (e.g., an optical component, the steering module 300, etc.). In a third example, a prism assembly (e.g., a biprism) can function to redirect unpatterned light (e.g., from the light source 100, from an optical component, etc.) onto the display module 200 and to redirect patterned light from the display module 200 to another system component (e.g., an optical component, the steering module 300, etc.).

In an example, the patterned light can be a set of patterned light frames (e.g., a time series of patterned light frames). The patterned light can optionally be defined by a set of light pattern parameters. For example, the light pattern parameters can prescribe a time series of patterned light frames, wherein one or more patterned light frames encode a frame of content. Each patterned light frame can optionally be produced by an array (e.g., two-dimensional array) of display elements. Each display element can include one or more optical components, actuators, and/or any other suitable elements. In a specific example, each display element can be or include a mirror. In another specific example, the display module 200 can include one or more actuators, wherein each actuator drives a display element. The array of display elements is preferably two-dimensional, but can alternatively be one-dimensional, three-dimensional, and/or any other number of dimensions. Each display element can be binary (e.g., on versus off; on-target versus deflected off-target; etc.), discrete (e.g., high intensity, low intensity, off), continuous, and/or otherwise configured.

Light pattern parameters can include global parameters defining the entire array of display elements and/or local parameters defining one or more individual display elements. In a specific example, a set of light pattern parameters can define each patterned light frame. Examples of global parameters and/or local parameters can include: origin location (e.g., location of the origin of the patterned light, location of a display element, etc.), patterned light direction (e.g., direction of the optical path of the projected patterned light, direction of the optical path of projected light from a display element, etc.), amplitude, phase, polarization, wavelength, spatial parameters (e.g., across the array of display elements), temporal parameters (e.g., a sequence of light pattern parameters defining a set of patterned light frames; a time associated with a display element being on or off; etc.), and/or any other light parameters. Light pattern parameters can be fixed (e.g., a fixed wavelength, fixed intensity, etc.) and/or variable.

Light pattern parameters can define the patterned light frame at the output of the display module 200 (e.g., an initial patterned light frame) and/or the patterned light frame projected on the target (e.g., a final patterned light frame). For example, an initial patterned light frame projected by the display module 200 can optionally undergo one or more adjustments prior to projection onto the target (e.g., adjustments made using optical components, supplemental light modulators, the steering module 300, etc.), such that a final patterned light frame projected onto the target (e.g., at the target location on the target) is different from the initial patterned light frame. In a specific example, the light pattern parameters defining the initial patterned light frame can be determined such that the final patterned light frame: is defined by a set of target light pattern parameters, is projected onto the target at the target location, is projected onto the target with a target orientation, and/or is otherwise configured.

The light pattern parameters can be determined based on measurements from the sensor module 400, a target location (e.g., wherein the target location is determined based on measurements from the sensor module 400), a target orientation (e.g., wherein the target orientation is determined based on measurements from the sensor module 400), a location of the projected patterned light (e.g., the most recent projected patterned light frame) on the target, an orientation of the projected patterned light (e.g., the most recent projected patterned light frame) on the target, content (e.g., a frame of content), current or previous light pattern parameters, current or previous steering module parameters, calibration information, a mapping of cells (e.g., RGCs) in the target, and/or any other suitable information. The light pattern parameters can be determined using the processing system 500, using the sensor module 400, using a model, manually, randomly, predetermined, and/or otherwise determined. In an example, the display module 200 (e.g., a spatial light modulator) can output patterned light based on the tracked location of an external feature of an eye and/or the tracked location of a retinal feature of the eye. In a specific example, the display module 200 (e.g., a spatial light modulator) can output patterned light based on the tracked location of the retinal feature. In another specific example, the display module 200 (e.g., a spatial light modulator) can output patterned light based on the tracked location of the external feature and the tracked location of the retinal feature.

However, the display module 200 can be otherwise configured.

The system can optionally include one or more supplemental light modulators (e.g., SLMs), which can function to adjust light pattern parameters of patterned light. The supplemental light modulators can optionally interface with and/or be part of: the display module 200, the steering module 300, and/or any other system component. In examples, the processing system 500 can control a supplemental light modulator (e.g., a phase modulator such as a liquid crystal phase modulator) based on measurements received by the sensor module 400, based on content, based on a cell mapping, manually, randomly, using a model, and/or otherwise controlled. In specific examples, the supplemental light modulator can be controlled based on eye gaze, calibration measurements, tracked features (e.g., tracked retinal features), and/or any other parameters. In an illustrative example, the supplemental light modulator can function to adjust patterned light such that the projected patterned light frame maps to corresponding cells in the target (e.g., accounting for an offset due to a change in eye gaze). In another illustrative example, the supplemental light modulator can function to achieve higher resolution imaging of the eye (e.g., to image individual cells). In another illustrative example, the supplemental light modulator can function to compensate for imperfections of the eye. However, supplemental light modulator(s) can be otherwise configured.

### 4.3. The Steering Module 300

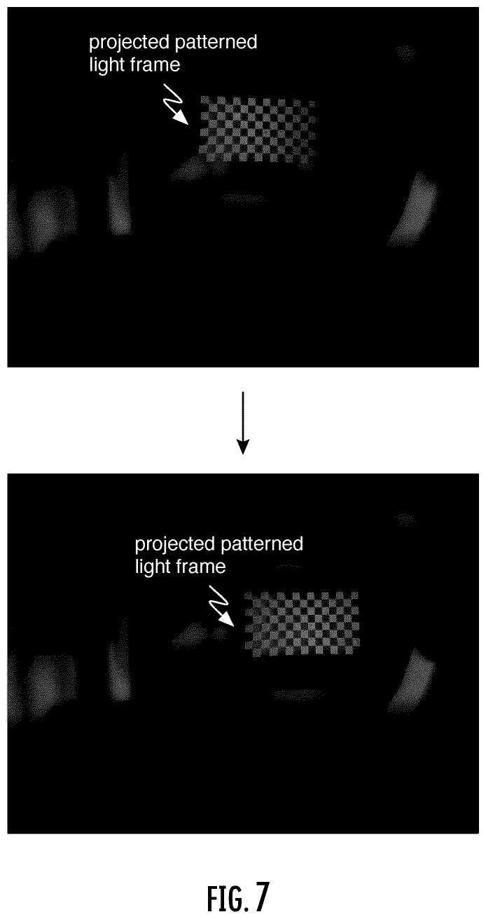

The steering module 300 functions to adjust the output light path (e.g., the optical path of patterned light projected onto the target) and/or adjust the pose of the patterned light frame projected on the target. In a specific example, the steering module 300 can steer the output light path to intersect with the pupil and/or the eyebox. In another specific example, the steering module 300 can steer the output light path to a target location on the target (e.g., a target location on the retina). In examples, the steering module 300 can adjust: the direction of the output light path, the origin of the output light path, the pose (e.g., position and/or orientation) of the patterned light frame on the target, one or more light pattern parameters of the output light, and/or otherwise adjust the output light and/or the resulting patterned light frame projected on the target. An example is shown in FIG. 7. The steering module 300 can receive light from the display module 200, receive light from one or more optical components, transmit light to the target, transmit light to one or more other optical components (e.g., to a focusing lens), and/or otherwise interface with one or more other system components.

The steering module 300 and/or a component thereof is preferably actuated, but can alternatively not be actuated. In examples, the steering module 300 and/or a component thereof can be actuated in one dimension, two dimensions, three dimensions, and/or more than three dimensions. In a first variant, the steering module 300 can include an actuated (e.g., active) optical component in the optical path of the light (e.g., in the optical path of unpatterned light before the display module 200 and/or in the optical path of the patterned light after the display module 200), which can adjust the output light path. In specific examples, the steering module 300 can include one or more actuated mirrors (e.g., single-axis mirror, two-axis mirror, etc.), actuated gratings, actuated spatial light modulators, actuated phase modulators, and/or any other actuated light modulator. In a second variant, the steering module 300 can include an actuated element of one or more system components (e.g., the light source 100, the display module 200, etc.), which can adjust the output light path. In a specific example, the display module 200 can modulate light pattern parameters (e.g., a different subset of display elements can be used, the direction of light from the display elements can be adjusted, etc.) to steer the output light. In a third variant, the steering module 300 can include an actuated component coupled to the target, which can adjust the pose of the target relative to the output light.

The steering module can optionally be controlled based on a set of steering module parameters (e.g., actuation of the steering module can be controlled based on the set of steering module parameters). In specific examples, steering module parameters can include: a target location, a target orientation, and/or any other information. In an example, the steering module 300 can direct a patterned light frame to a location at or near the target location. The steering module parameters can be determined based on measurements from the sensor module 400, a target location (e.g., wherein the target location is determined based on measurements from the sensor module 400), a target orientation (e.g., wherein the target orientation is determined based on measurements from the sensor module 400), a location of the projected patterned light (e.g., the most recent projected patterned light frame) on the target, an orientation of the projected patterned light (e.g., the most recent projected patterned light frame) on the target, content, a mapping of cells in the target, current or previous light pattern parameters, current or previous steering module parameters, calibration information, and/or any other suitable information. The steering module parameters can be determined using the processing system 500, using the sensor module 400, using a model, manually, randomly, predetermined, and/or otherwise determined. In a specific example, the steering module 300 can steer the patterned light based on the tracked location of an external feature of the eye and/or the tracked location of a retinal feature of the eye. In a specific example, the steering module 300 can steer the patterned light based on the tracked location of the external feature. In another specific example, the steering module 300 can steer the patterned light based on the tracked location of the external feature and the tracked location of the retinal feature.

However, the steering module 300 can be otherwise configured.

### 4.4. The Sensor Module 400

The sensor module 400 functions to: image and/or track all or a portion of the target (e.g., a retinal feature), image and/or track all or a portion of an external feature of the eye (e.g., the pupil), image and/or track the patterned light frame on the target, provide feedback for one or more system components (e.g., the display module 200, the steering module 300, the retinal implant 600), and/or collect any other measurements. The sensor module 400 can transmit measurements to the processing system 500, and/or otherwise interface with one or more system components.

The sensor module 400 and/or components therein can optionally be actuated (e.g., in 1 dimension, in 2 dimensions, 3 dimensions, etc.). The sensor module 400 can include one or more sensors (e.g., at least two sensors, at least three sensors, etc.). In an example, the sensor module can include a first sensor (e.g., external feature sensor) configured to collect a set of measurements (e.g., images) of an eye (e.g., of an external feature of an eye) and a second sensor (e.g., retinal feature sensor) configured to collect a set of measurements (e.g., images) of a retina of the eye (e.g., of a retinal feature of the retina of the eye). In a specific example, the first sensor (e.g., external feature sensor) can be configured to receive light having a first wavelength (e.g., produced using a first light source and reflected off of the eye) and the second sensor (e.g., retinal feature sensor) can be configured to receive light having a second wavelength (e.g., produced using a second light source and reflected off of a retinal feature; fluorescent light produced by retinal cells, in response to absorbing light from a second light source; etc.).
the set of measurements of the eye of the user is sampled by the first sensor using light from a first light source projecting light at a first wavelength, wherein the set of measurements of the retina is sampled by the second sensor using light from a second light source projecting light at a second wavelength.

Examples of sensors in the sensor module 400 can include: light sensor (e.g., camera, photodiode, etc.), motion sensor (e.g., inertial measurement unit, retroreflector, other optical components, etc.), depth sensor, thermal sensor, electrodes, and/or any other sensor. The light sensor can measure IR, visible light (e.g., RGB), UV light, and/or any other wavelength. In a first specific example, the sensor module 400 can include one or more cameras (e.g., full-field camera, scanning cameras, etc.). In a second specific example, the sensor module 400 can include one or more photodiodes. The sensor module 400 can optionally include one or more light sources (e.g., LEDs, laser, any light source as described above, etc.). The sensor module 400 can optionally include one or more optical components.

In variants, the sensor module 400 can include a light source and a sensor (e.g., a camera). The light source can project light onto a sensor target (e.g., an eye, a retina, etc.), and the sensor can receive light from the sensor target. The sensor target can be the same as the target for the projector and/or can be a different target. In an example, the light source can be the output light from the projector or a separate light source.

In a first variant, the sensor module 400 can include an external feature sensor (e.g., an external eye camera), which can be used to image and/or track one or more external eye features. Examples of external eye features include: the pupil (e.g., the center of the pupil), corneal reflection, and/or any other eye features. In a specific example, light from a light source (e.g., the light source 100 or a separate light source) can reflect off of the external eye feature(s) to the external feature sensor (e.g., directly or via one or more optical components).

In a second variant, the sensor module 400 can include a retinal feature sensor (e.g., a retinal camera such as a fundus camera), which can be used to image and/or track one or more retinal features. In a first embodiment, retinal features can include visual features of the retina (e.g., one or more individual cells, vasculature, visual streaks, optic disk, fluorescent markers, etc.) and/or visual features of the retinal implant 600 (e.g., the center of the retinal implant 600, pixels of the retinal implant 600, etc.). In a specific example, fluorescence of cells (e.g., genetically modified cells and/or non-modified cells) in the target can be imaged and/or tracked, wherein a fluorescent protein in the cells can be stimulated using a light source (e.g., the light source 100 or a separate light source). In another specific example, light from a light source (e.g., the light source 100 or a separate light source) can reflect off of the retinal feature(s) to the retinal feature sensor (e.g., via a dichroic and/or any other optical component). In a second embodiment, the retinal features can include visual features of the patterned light frame on the retina and/or the retinal implant 600. In a specific example, the patterned light frame can reflect light back from the retina to the retinal feature sensor (e.g., via a dichroic and/or any other optical component).

In an example, a dichroic can be used to direct light having a first wavelength (e.g., the patterned light) to the target (e.g., wherein the light having the first wavelength enters the dichroic from a first direction) along a first optical path and to direct light having a second wavelength (e.g., light from a sensor module light source, optionally via one or more optical components) to the target (e.g., entering the dichroic from a second direction) along a second optical path. In a first specific example, the dichroic can direct reflected light from the target (e.g., light having the second wavelength, reflected off a retinal feature) to the retinal feature sensor (e.g., along a path opposite to the second optical path). In a second specific example, the dichroic can direct fluorescent light from the target (e.g., light having a third wavelength, generated by retinal cells) to the retinal feature sensor.

In a third variant, a combination of the previous embodiments can be used (e.g., the sensor module 400 can include both an external feature sensor and a retinal feature sensor).

Measurements acquired using the sensor module 400 can optionally be used for diagnostics. For example, measurements of the retina (e.g., acquired using the retinal feature sensor) can be used to determine a retinal health parameter (e.g., an evaluation of the health of the retina and/or cells therein). In a first embodiment, retinal images (e.g., fundus images) acquired using the retinal feature sensor can be used to evaluate the cells of the retina. In a first specific example, the retinal images can be used to evaluate optogenetically modified cells in the retina (e.g., determining which cells were successfully transfected with the target gene, monitoring the production of the opsin over time, etc.). In a second specific example, the retinal images can be used to perform fundus autofluorescence imaging, wherein retinal images (e.g., acquired using the retinal feature sensor) can be used to monitor fluorophores in the retina for evaluation of the health of the retina. In a second embodiment, retinal images acquired using the retinal features sensor can be used to evaluate the retinal implant. In an illustrative example, the retinal images can be used to visualize the retinal implant to monitor the location of the retinal implant on the retina.

However, the sensor module 400 can be otherwise configured.

### 4.5. The Processing System 500

The processing system 500 functions to control the light source 100, control the display module 200 (e.g., determining the light pattern parameters and controlling the display module 200 based on the light pattern parameters), control the steering module 300 (e.g., determining the steering module parameters and controlling the steering module 300 based on the steering module parameters), and/or otherwise control one or more system components. The processing system 500 can optionally receive measurements (e.g., images and/or other data) from the sensor module 400 (e.g., from one or more sensors in the sensor module 400).

The processing system 500 can include or use one or more models, including one or more rendering models, light propagation models, tracking models, and/or any other model.

The models can include classical or traditional approaches, machine learning approaches, and/or be otherwise configured. The models can include regression, decision tree, clustering, association rules, dimensionality reduction, language processing techniques (e.g., LSA), neural networks (e.g., GNN, CNN, DNN, CAN, LSTM, RNN, FNN, encoders, decoders, deep learning models, transformers, etc.), ensemble methods, optimization methods, classification, rules, heuristics, equations (e.g., weighted equations, etc.), selection, lookups, regularization methods (e.g., ridge regression), Bayesian methods (e.g., Naiive Bayes, Markov, etc.), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, foundation models (e.g., language models), computer vision models (e.g., feature extractors, segmentation models, object detectors, etc.), and/or any other suitable model. Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated.

The processing system 500 can optionally determine light pattern parameters based on content. The content is preferably visual content, but can alternatively be any other content (e.g., content for other senses, such as temperature, other information not related to senses, etc.). Examples of content include: measurements (e.g., images, videos, etc.) of a real-world scene; artificial reality (AR) overlays; AR overlaid onto measurements of a real-world scene; virtual reality (VR); text; and/or any other information. In an example, the content can be a set of images captured using a camera. In a specific example, the patterned light can be a time series of patterned light frames, wherein each patterned light frame corresponds to an image (e.g., a frame) of the set of images. In specific examples, the image can be a processed image (e.g., cropped, downsampled, etc.) or an unprocessed image. In an example, the rendering model can output light pattern parameters based on content. In a specific example, the rendering model can output light pattern parameters for one or more patterned light frames based on a content frame.

The processing system 500 can optionally detect and/or track a location of one or more target features. In examples, target features include external features of the eye (e.g., as described above), retinal features (e.g., as described above), and/or any other feature associated with the target. The location of the target feature(s) can be used to control: the steering module 300 (e.g., to adjust steering module parameters), the display module 200 (e.g., to adjust light pattern parameters), supplemental light modulator(s), to calibrate one or more system components, and/or any other system component. In an example, the processing system 500 can be configured to: track a location of the external feature based on the set of measurements of the external feature (e.g., collected via the sensor module 400); track a location of the retinal feature based on the set of measurements of the retinal feature (e.g., collected via the sensor module 400); control the steering module 300 based on the tracked location of the external feature and/or the tracked location of the retinal feature; and control the display module 200 (e.g., spatial light modulator) based on the tracked location of the external feature and/or the tracked location of the retinal feature. In a specific example, the processing system 500 can be configured to control the steering module 300 (e.g., to steer the patterned light) based on the tracked location of the external feature. In another specific example, the processing system 500 can be configured to control the steering module 300 (e.g., to steer the patterned light) based on the tracked location of the external feature and the tracked location of the retinal feature. In a specific example, the processing system 500 can be configured to control the display module 200 (e.g., to pattern light) based on the tracked location of the retinal feature. In another specific example, the processing system 500 can be configured to control the display module 200 (e.g., to pattern light) based on the tracked location of the external feature and the tracked location of the retinal feature.

In a first variant, the processing system 500 can detect and/or track a location of one or more external features of the eye (e.g., pupil, corneal reflection, etc.). For example, the location of the external feature(s) can be used to track a user's gaze. The location of the external feature(s) can optionally be detected and/or tracked based on measurements collected using the sensor module 400 (e.g., the external feature sensor). In an example, the location of the external feature(s) can be used to steer the output light using the steering module 300. For example, a first target location (e.g., an approximate target location) can be determined based on the location of the external feature, wherein the steering module 300 can be controlled to direct the patterned light onto the target towards the first target location. In a specific example, the steering module 300 can be controlled to direct the patterned light onto the target within a threshold distance of the first target location (e.g., within 1000 nm, within 500 nm, within 200 nm, etc.).

In a second variant, the processing system 500 can detect and/or track a location of one or more retinal features (e.g., an individual retinal cell, vasculature, features of the retinal implant 600, features of the projected light frame, etc.). The location of the retinal feature(s) can optionally be detected and/or tracked based on measurements collected using the sensor module 400 (e.g., the retinal feature sensor). Retinal features can optionally be tracked at a first sampling rate, while external eye features are tracked at a second sampling rate.

In a first embodiment of the second variant, the location of the retinal feature(s) can be used to steer the output light using the steering module 300. In a specific example, the tracked retinal feature(s) can be used to supplement tracked external eye feature(s), to increase the resolution of eye and/or gaze tracking. For example, an updated target location (e.g., a precise target location) can be determined based on the location of the external feature, wherein the steering module 300 can be controlled to direct the patterned light onto the target towards the updated target location. In a specific example, the steering module 300 can be controlled to direct the patterned light onto the target within a threshold distance of the updated target location (e.g., within 1000 nm, within 500 nm, within 200 nm, etc.).

In a second embodiment of the second variant, the location of the retinal feature(s) can be used to determine the light pattern parameters (e.g., to adjust the light pattern parameters). In a specific example, the tracked location of the retinal feature(s) can be used to perform a pose adjustment (e.g., adjusting orientation, adjusting scale, adjusting location, other affine transformations, etc.) of the light pattern relative to the retinal features. In an illustrative example, the light pattern parameters can be used to adjust for system aberrations and/or aberrations of the eye. In an example, an updated target location (e.g., a precise target location) can be determined based on the location of the external feature, wherein the display module 200 can be controlled to adjust the patterned light parameters such that the patterned light is projected onto the target at or near the updated target location. In a specific example, the display module 200 can be controlled to adjust the patterned light parameters such that the patterned light is projected onto the target within a threshold distance of the updated target location (e.g., within 200 nm, within 100 nm, within 50 nm, within 25 nm, etc.).

In a third embodiment of the second variant, the location of the retinal feature(s) can be used to determine a mapping of cells in the target. The cell mapping can include coordinates of individual target cells, a mapping between a patterned light frame and the target cells, a retinal coding scheme (e.g., a spatial mapping of classifications of retinal ganglion cell types in the retina), a mapping of cell function, and/or any other cell information. In a specific example, light pattern parameters for a light modulator (e.g., for the display module 200 and/or a supplement light modulator) can be determined based on the cell mapping. In an illustrative example, each element of the projected patterned light frame (e.g., driven by a display element of the light modulator) can map to one or more cells in the target, wherein light pattern parameters can be determined such that each element of the projected patterned light frame is projected onto the corresponding cell(s). In variants, the patterned light frame projected onto the target can be a grayscale image, wherein the brain interprets the projected patterned light frame (and/or a series of patterned light frames) as a color image. In specific examples, the cell mapping can be used to adjust the light pattern parameters such that one or more elements of the patterned light frame that corresponds to a color (e.g., encodes a color) stimulates target cell(s) corresponding to that color and/or stimulates target cell(s) such that the brain interprets the stimulation as that color.

In a fourth embodiment of the second variant, the retinal feature(s) can be used to determine to calibrate one or more system components (e.g., calibrating the light pattern parameters and/or the steering module parameters).

In variants, a tracking model can output tracked coordinates of a target feature, a tracked gaze, and/or any other suitable outputs. Inputs to the tracking model can include measurements (e.g., images) received from the sensor module 400. In examples, the tracking model can use a deep-learning based approach (e.g., using an NN to segment an image frame, and post-processing the segmented image to determine a pupil contour), a computer-vision approach (e.g., pre-processing an image frame to generate a binary image, and using blob detection to determine a pupil contour), and/or any other image analysis methods. Outputs from the tracked model can optionally be processed. Examples of processing include filtering, affine transformation, scaling, and/or any other processing methods. In a first example, the tracking model can output coordinates of the pupil. In a second example, the tracking model can output coordinates of the corneal reflection. In a third example, the tracking model (and/or a downstream model from the tracking model) can output a tracked gaze vector, wherein the gaze vector is based on a vector between pupil coordinates and corneal reflection coordinates.

However, the processing system 500 can be otherwise configured.

### 4.6. Retinal Implant 600

The system can optionally include a retinal implant 600, which functions to emit excitation signals that are received by retinal cells (e.g., genetically modified cells and/or non-modified cells), in response to receiving light from the projector. The excitation signals (e.g., electrical signals) can encode content (e.g., visual data, sensory data, other external information, artificial data, any other data, etc.).

The retinal implant 600 can be implanted on or below the retina. In specific examples, the retinal implant 600 can be a subretinal implant or an epiretinal implant. In specific examples, the retinal implant can include systems as described in U.S. application Ser. No. 18/740,854 filed 12 Jun. 2024, which is incorporated in its entirety by this reference.

The retinal implant 600 can include one or more pixels, wherein each pixel includes circuitry and/or components configured to receive a light signal (e.g., a portion of a patterned light frame) and emit excitation signals (e.g., electrical signals) in response to receiving the light signal. In specific examples, pixels can include photovoltaic cells, photodiodes, any light sensor system, any electrical emission system (e.g., electrodes), any light emission system (e.g., μLEDs, etc.), and/or any other optical components. In a specific example, the retinal implant 600 can operate at or near cellular resolution (e.g., one pixel can excite less than 20 cells, less than 10 cells, less than 5 cells, less than 2 cells, 1 cell, etc.). The number of pixels can be between 100-50,000 or any range or value therebetween (e.g., at least 1000; at least 2000; at least 5000; at least 10000; etc.), but can alternatively be less than 100 or greater than 50,000.

The retinal implant 600 can optionally include and/or be coupled (e.g., adhered, mounted, etc.) to one or more optics components (e.g., microoptical components). The optics component can function to collimate light, homogenize light, focus light (e.g., onto the light sensor systems), reduce light shining back through the eye lens, and/or otherwise modify light emitting to or from the display 100. The optics component can be located on the front of the retinal implant 600 (facing the retina) and/or back of the retinal implant 600. Examples of optics components can include lenses (e.g., microlens, diffractive lens, metalens, etc.), back reflectors, mirrors, light modulators, diffractive components, diffusers, filters, fibers, waveguides, prisms, dichroics, irises/apertures, and/or any other optical components.

However, the retinal implant 600 can be otherwise configured.

### 5. Specific Examples

A numbered list of specific examples of the technology described herein are provided below. A person of skill in the art will recognize that the scope of the technology is not limited to and/or by these specific examples.
Specific Example 1. A system, comprising: a light source configured to project light; a spatial light modulator configured to modulate the light to output patterned light; a steering module configured to steer the patterned light; a first sensor configured to collect a set of measurements of an external feature of an eye; a second sensor configured to collect a set of measurements of a retinal feature of a retina of the eye; and a processing system configured to: track a location of the external feature based on the set of measurements of the external feature; track a location of the retinal feature based on the set of measurements of the retinal feature; control the steering module based on the tracked location of the external feature; and control the spatial light modulator based on the tracked location of the retinal feature.
Specific Example 2. The system of Specific Example 1, wherein the external feature comprises at least one of a pupil or a corneal reflection.
Specific Example 3. The system of any of Specific Examples 1 or 2, wherein the retinal feature comprises at least one of an individual retinal cell or retinal vasculature.
Specific Example 4. The system of any of Specific Examples 1-3, wherein the retinal feature comprises a feature of a retinal implant.
Specific Example 5. The system of any of Specific Examples 1-4, wherein the processing system is configured to control the steering module further based on the tracked location of the retinal feature.
Specific Example 6. The system of any of Specific Examples 1-5, wherein the light source is configured to project light having a first wavelength, the system further comprising a second light source configured to project light having a second wavelength, wherein the second sensor is configured to receive light having the second wavelength.
Specific Example 7. The system of Specific Example 6, further comprising a dichroic, wherein the dichroic is configured to direct the patterned light to the retina and direct light originating from the second light source to the retina.
Specific Example 8. The system of Specific Example 6, further comprising a third light source configured to project light having a third wavelength, wherein the first sensor is configured to receive light having the third wavelength.
Specific Example 9. The system of any of Specific Examples 1-8, wherein the steering module is configured to steer the patterned light to a target region of the retina comprising genetically modified cells, wherein the genetically modified cells are transfected with a gene for a light-sensitive protein, wherein the genetically modified cells produce biochemical signals in response to receiving the patterned light.
Specific Example 10. The system of any of Specific Examples 1-9, further comprising a retinal implant, wherein the steering module is configured to steer the patterned light to the retinal implant, wherein the retinal implant is configured to produce electrical signals in response to receiving the patterned light.
Specific Example 11. A method, comprising: using a first sensor, sampling a set of measurements of an eye of a user; using a second sensor, sampling a set of measurements of a retina of the eye of the user; tracking a location of an external feature of the eye based on the set of measurements of the eye; tracking a location of a retinal feature of the retina based on the set of measurements of the retina; using a spatial light modulator, producing patterned light based on content and the tracked location of the retinal feature; and steering the patterned light based on the tracked location of the external feature.
Specific Example 12. The method of Specific Example 11, wherein the retinal feature comprises at least one of an individual retinal cell or retinal vasculature.
Specific Example 13. The method of any of Specific Examples 11 or 12, wherein the retinal feature comprises at least one of individual retinal cells or vasculature.
Specific Example 14. The method of any of Specific Examples 11-13, wherein the content comprises a set of images, wherein the patterned light comprises a time series of patterned light frames, wherein each patterned light frame corresponds to an image of the set of images.
Specific Example 15. The method of any of Specific Examples 11-14, wherein the patterned light is further steered based on the tracked location of the retinal feature.
Specific Example 16. The method of Specific Example 15, wherein steering the patterned light comprises: determining a first target location based on the tracked location of the external feature; steering the patterned light towards the first target location; determining an updated target location based on the tracked location of the retinal feature; and steering the patterned light towards the updated target location.
Specific Example 17. The method of any of Specific Examples 11-16, wherein the set of measurements of the eye of the user is sampled by the first sensor using light from a first light source projecting light at a first wavelength, wherein the set of measurements of the retina is sampled by the second sensor using light from a second light source projecting light at a second wavelength.
Specific Example 18. The method of any of Specific Examples 11-17, wherein the second sensor samples the set of measurements of the retina using light reflected off the retinal feature.
Specific Example 19. The method of any of Specific Examples 11-18, wherein the second sensor samples the set of measurements of the retina using fluorescent light from retinal cells.
Specific Example 20. The method of any of Specific Examples 11-19, further comprising determining a retinal health parameter based on the set of measurements of the retina.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

All references cited herein are incorporated by reference in their entirety, except to the extent that the incorporated material is inconsistent with the express disclosure herein, in which case the language in this disclosure controls.

Different subsystems and/or modules discussed above can be defined, operated, and/or controlled by the same or different entities. In the latter variants, different subsystems can communicate via: APIs (e.g., using API requests and responses, API keys, etc.), requests, and/or other communication channels.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

1. A system, comprising:
a light source configured to project light;
a spatial light modulator configured to modulate the light to output patterned light;
a steering module configured to steer the patterned light;
a first sensor configured to collect a set of measurements of an external feature of an eye;
a second sensor configured to collect a set of measurements of a retinal feature of a retina of the eye; and
a processing system configured to:
track a location of the external feature based on the set of measurements of the external feature;
track a location of the retinal feature based on the set of measurements of the retinal feature;
control the steering module based on the tracked location of the external feature; and
control the spatial light modulator based on the tracked location of the retinal feature.

2. The system of claim 1, wherein the external feature comprises at least one of a pupil or a corneal reflection.

3. The system of claim 1, wherein the retinal feature comprises at least one of an individual retinal cell or retinal vasculature.

4. The system of claim 1, wherein the retinal feature comprises a feature of a retinal implant.

5. The system of claim 1, wherein the processing system is configured to control the steering module further based on the tracked location of the retinal feature.

6. The system of claim 1, wherein the light source is configured to project light having a first wavelength, the system further comprising a second light source configured to project light having a second wavelength, wherein the second sensor is configured to receive light having the second wavelength.

7. The system of claim 6, further comprising a dichroic, wherein the dichroic is configured to direct the patterned light to the retina and direct light originating from the second light source to the retina.

8. The system of claim 6, further comprising a third light source configured to project light having a third wavelength, wherein the first sensor is configured to receive light having the third wavelength.

9. The system of claim 1, wherein the steering module is configured to steer the patterned light to a target region of the retina comprising genetically modified cells, wherein the genetically modified cells are transfected with a gene for a light-sensitive protein, wherein the genetically modified cells produce biochemical signals in response to receiving the patterned light.

10. The system of claim 1, further comprising a retinal implant, wherein the steering module is configured to steer the patterned light to the retinal implant, wherein the retinal implant is configured to produce electrical signals in response to receiving the patterned light.

11. A method, comprising:
using a first sensor, sampling a set of measurements of an eye of a user;
using a second sensor, sampling a set of measurements of a retina of the eye of the user;
tracking a location of an external feature of the eye based on the set of measurements of the eye;
tracking a location of a retinal feature of the retina based on the set of measurements of the retina;
using a spatial light modulator, producing patterned light based on content and the tracked location of the retinal feature; and
steering the patterned light based on the tracked location of the external feature.

12. The method of claim 11, wherein the external feature comprises at least one of a pupil or a corneal reflection.

13. The method of claim 11, wherein the retinal feature comprises at least one of an individual retinal cell or retinal vasculature.

14. The method of claim 11, wherein the content comprises a set of images, wherein the patterned light comprises a time series of patterned light frames, wherein each patterned light frame corresponds to an image of the set of images.

15. The method of claim 11, wherein the patterned light is further steered based on the tracked location of the retinal feature.

16. The method of claim 15, wherein steering the patterned light comprises:
determining a first target location based on the tracked location of the external feature;
steering the patterned light towards the first target location;
determining an updated target location based on the tracked location of the retinal feature; and
steering the patterned light towards the updated target location.

17. The method of claim 11, wherein the set of measurements of the eye of the user is sampled by the first sensor using light from a first light source projecting light at a first wavelength, wherein the set of measurements of the retina is sampled by the second sensor using light from a second light source projecting light at a second wavelength.

18. The method of claim 11, wherein the second sensor samples the set of measurements of the retina using light reflected off the retinal feature.

19. The method of claim 11, wherein the second sensor samples the set of measurements of the retina using fluorescent light from retinal cells.

20. The method of claim 11, further comprising determining a retinal health parameter based on the set of measurements of the retina.
